(19) United States
(12) Patent Application Publication
(10) Pub. No.: US 2026/0174841 A1
(43) Pub. Date: Jun. 25, 2026

(54) METHODS AND COMPOSITIONS OF QUATERNARY IMMUNOMODULATORY NANOPARTICLES FOR MEDICAL APPLICATIONS AND IMMUNOTHERAPY

(71) Applicant: Astrid Pharma Corp., Davis, CA (US)

(72) Inventors: R. Holland CHENG, Davis, CA (US); Chun-Chieh CHEN, Winters, CA (US); Chin-Fu CHEN, Clemson, CA (US)

(21) Appl. No.: 18/874,623

(22) PCT Filed: Jun. 16, 2023
(86) PCT No.: PCT/US2023/068570

§ 371 Date: Dec. 13, 2024

Related U.S. Application Data
(60) Provisional application No. 63/352,945, filed on Jun. 16, 2022.

(51) Int. Cl. A61K 39/12; A61K 9/14; A61K 39/00; A61P 37/04

(52) U.S. Cl. CPC A61K 39/12; A61K 9/14; A61K 39/0002; A61P 37/04; A61K 2039/55516

(57) ABSTRACT

The present disclosure provides a method of enhancing an immune response in a subject in need thereof, including administering an effective amount of an antigen with an immune modulator to the subject, wherein the immune modulator is a quaternary immunomodulatory nanoparticle (QIN). The present disclosure also provides the use of the QIN in the manufacture of a chimeric nanoparticle, a pharmaceutical composition and a medicament for treating a subject suffering from a cancer or virus infection.

### CROSS REFERENCE TO RELATED APPLICATIONS

This application is a national stage application of PCT/US2023/068570, filed on Jun. 16, 2023, which claims the benefit under 35 U.S.C. § 119 (e) of U.S. Provisional Patent Application Ser. No. 63/352,945, filed on Jun. 16, 2022, the entirety of which is incorporated herein by reference.

### REFERENCE TO SEQUENCE LISTING

Incorporated by reference in its entirety herein is a computer-readable nucleotide/amino acid sequence listing submitted concurrently herewith and identified as follows: One 6,038 Byte XML file named “23P0339_PCT_Sequence listing_F1-20230616-toAgent.xml,” created on Dec. 11, 2024.

### FIELD OF INVENTION

The present disclosure relates to quaternary immunomodulatory nanoparticles (QINs), derived from hepatitis E virus nanoparticles, particularly the use of QINs in enhancing an immune response in a subject and the manufacture of a chimeric nanoparticle, a pharmaceutical composition, and a medicament.

### BACKGROUND OF THE INVENTION

Hepatitis E is an enteric-transmitted virus that causes acute liver inflammation in humans. Its derivative quaternary immunomodulatory nanoparticle (QIN) based on Hepatitis E virus nanoparticle displays an icosahedral cage that is stable in acid and proteolytic environments, a feature that is retained from its natural transmission route.

Hepatitis E virus is composed of a non-enveloped icosahedral capsid enclosing a single-stranded RNA genome of 7.2 kilobases and is an enteric-transmitted virus that causes acute liver inflammation in humans. A major capsid protein is essential for virus assembly, immunogenicity, and host interaction. The recombinant capsid protein can self-assemble into virus-like particles when expressed in insect cells after the deletion of 111 amino acids from the N-terminal end and 52 amino acids from the C-terminal end. The QIN displays an icosahedral cage that is stable in acid and proteolytic environments and has a feature retained from its natural transmission route. The QIN consists of 60 subunits and forms an icosahedral nanoparticle. The QIN is stable in an acidic environment and resistant to proteolytic digestion, and thus it poses a great advantage as an oral delivery vehicle.

A natural fungal medicine derived from traditional Chinese medicine, Ganoderma, also called Lingzhi, is one of the most well-known Chinese traditional medicinal species. It has been widely used to treat various diseases in China, America, Japan, Korea, and other Asian countries for centuries. Its immunomodulatory protein and polysaccharides have been identified as key bioactive factors for cancer immunotherapy, and the NF-κB and MAPK pathways. Thus, even in modern medicine practice, Ganoderma has been used to treat chronic bronchitis, bronchial asthma, leukopenia, coronary heart disease, arrhythmia, and acute infectious hepatitis. However, it is mainly used as supplementary treatment in addition to first-line therapy in the current clinical setting. Many studies have indicated that Ganoderma has an antitumor effect caused by regulating the immune system, attributed to its fungal immunomodulation proteins (FIPs), polysaccharides, and triterpenoids. Among them, GMI, an important immunomodulatory protein cloned from Ganadorma microsporum, has been studied intensely. In vitro test performed by Lin et al., it was found that GMI inhibited the EGF-induced phosphorylation and activation of EGFR and AKT pathway kinases in a dose-dependent manner. In vivo studies also suggested that the oral administration of GMI inhibited tumor growth and induced autophagy in mice models.

Porcine circovirus type 2 (PCV2) is a small, non-enveloped DNA virus, which is recognized as one of the most significant swine pathogens of porcine circovirus disease (PCVD), causing economic losses and reduced productivity in all major swine-producing countries around the world. Vaccination against PCV2 is now a routine practice in the swine sector of most developed countries. A commercially available PCV2 subunit vaccine, CIRCOQ, is developed by Reber Genetics Company (Taipei, Taiwan) to protect against emerging PCV2.

### SUMMARY OF THE INVENTION

The present invention provides medical applications of quaternary immunomodulatory nanoparticles (QINs) and, in particular, the use of QINs in enhancing an immune response in a subject suffering from cancer or virus infection and in manufacturing a chimeric nanoparticle, a pharmaceutical composition, and a medicament.

In an aspect of the present invention, a method of enhancing an immune response in a subject in need thereof is provided, and the method includes administering an effective amount of an antigen with an immune modulator to the subject, wherein the immune modulator is a quaternary immunomodulatory nanoparticles (QIN).

Preferably, the QIN is further coupled with an epitope of a Ganoderma microsporum immunomodulatory (GMI) protein.

Preferably, the epitope of the GMI protein is coupled with the QIN by inserting into a capsid subunit of the QIN.

Preferably, the epitope of the GMI protein has a sequence represented by SEQ ID NO: 1

Preferably, the antigen is selected from a group consisting of a tumor antigen, a virus antigen, a bacterial antigen, an autoantigen, and a fungal antigen.

Preferably, the virus antigen is a PCV2 antigen.

In another aspect of the present invention, a chimeric nanoparticle including a QIN coupled with an epitope of a GMI protein is provided.

In further another aspect of the present invention, a pharmaceutical composition including a QIN and a pharmaceutically acceptable carrier, vehicle, or diluent is provided.

In yet another aspect of the present invention, a use of the pharmaceutical composition described above in the manufacture of a medicament for treating a subject suffering from a cancer or virus infection is provided.

### BRIEF DESCRIPTION OF THE DRAWINGS

FIG. 1a illustrates the expression of 22 selected genes affected by QIN in qPCR analysis with QIAGEN plates.

FIG. 1b illustrates the expression of 4 selected genes affected by QIN in individual qPCR analysis.

FIG. 2 illustrates among all selected genes expression affected by QIN, the relationship of 13 selected genes interacted with each other shown in the generated Protein-Protein Interaction Network.

FIG. 3 illustrates Protein-Protein Interaction Network expanded from the 13 genes stimulated by QIN in FIG. 2.

FIG. 4a illustrates the expression of 11 selected genes affected by QIN and QIN-GMI in qPCR analysis with QIAGEN plates.

FIG. 4b illustrates the expression of 3 selected genes affected by QIN and QIN-GMI in individual qPCR analysis.

FIG. 5 illustrates the expression of 22 selected genes affected by GMI, QIN and QIN-GMI in qPCR gene analysis with QIAGEN plates.

FIG. 6 illustrates the animal test results using QIN as an immune modulator (QIN-IM) in pigs vaccinated with the CIRCOQ vaccine.

### DETAILED DESCRIPTION

The following representative examples illustrate various features and embodiments of the disclosure, intended to be illustrative and not limiting. Those skilled in the art will readily appreciate that the specific examples are only illustrative of the invention as described more fully in the subsequent claims. Every embodiment and feature described in the present application should be understood to be interchangeable and combinable with every embodiment contained within.

Unless defined otherwise, all technical and scientific terms and any acronyms used herein have the same meanings as commonly understood by one of ordinary skill in the art in the field of this invention. All publications cited herein are incorporated by reference.

In an embodiment, a method of enhancing an immune response in a subject in need thereof includes administering an effective amount of an antigen with an immune modulator to the subject, wherein the immune modulator is a quaternary immunomodulatory nanoparticles (QIN).

An embodiment provides a chimeric nanoparticle including a QIN coupled with an epitope of a GMI protein.

An embodiment provides a pharmaceutical composition including a QIN and a pharmaceutically acceptable carrier, vehicle, or diluent.

In an embodiment, the pharmaceutical composition described above is provided to manufacture a medicament for treating cancer or virus infection subjects.

The QIN is coupled with an epitope of a Ganoderma microsporum immunomodulatory (GMI) protein in an embodiment. In an embodiment, the QIN may be orally administered to the subject. In another embodiment, the subject may be an animal species selected from the mouse, rat, rabbit, dog, pig, sheep, cow, and primate group. In another embodiment, the antigen may be selected from a group consisting of a tumor antigen, a virus antigen, a bacterial antigen, an autoantigen, and a fungal antigen.

### EXAMPLES

### Materials and Methods

### Cell Culture:

THP-1 cells were purchased from the American Type Culture Collection (ATCC). The cells were cultured in RPMI-1640 medium, supplemented with 10% FBS in 6% CO₂, 37° C. incubator. The cells were passed the day before treatment with the concentration of 5×10⁵/ml. The cells were harvested 48 hours after treatment for RNA extraction and the supernatants for cytokine ELISA analysis.

RNA and cDNA:

RNA was purified from the treated cells with the RNeasy Mini Kit (Qiagen 74104). 0.5 μg of RNA was reverse transcribed with SuperScript™ III First-Strand Synthesis SuperMix for qRT-PCR (Thermofisher 11752250) to get cDNA to be used in RT-PCR.

### RT-PCR:

Predesigned, species-specific and gene specific KiCqStart™ Primers from Sigma were used. RT-PCR was performed by Luminaris Color HiGreen qPCR Master Mix (Thermofisher K0392). The final concentration of each forward and reverse primer was 0.3 mM, and reaction volume was 20 ml. Three-step cycling protocol was followed. The reaction started with 50° C., 2 min, 95° C. 10 min followed by 40 cycles of 95° C., 15 sec, 60° C., 30 sec, and 72° C., 30 sec. PCR was done with CFX96 Real-Time PCR Detection System from Bio-Rad.

### ELISA:

The human inflammatory cytokines in the supernatants were analyzed by Multi-Analyte ELISArray Kit (Qiagen MEH-004A). The cytokines represented by this array are IL1a, IL1b, IL2, IL4, IL6, IL8, IL10, IL12, IL17A, IFNg, TNFa, and GM-CSF. The 96-well ELISA plate has been coated with a panel of cytokine target-specific antibodies, one in each 8-well strip. Standard ELISA procedures were followed to detect the cytokines in each sample. Positive and negative controls were included in the assay.

### Building Construct of QIN

The coding sequence of QIN, dORF2 (obtained from e.g., but not limited to, crystal structure analysis of the capsid protein [Hepatitis E virus (strain Myanmar)] Sequence ID: 2ZZQ_A (SEQ ID NO: 1); other known sequences of second open reading frame (ORF2) also can be used herein, such as those described in the U.S. patent No. US20190031720 A1), was modified as the sequence represent by SEQ ID NO: 2 to meet the best expression efficiency in insect cells, and then synthesized by Integrated DNA Technologies (Skokie, IL). The pFastBac1-dORF2 was produced and used to generate the recombinant baculovirus AcBactoBac-dORF2 following the Bac-to-Bac® Baculovirus Expression System kit protocol (Life Technologies). Similarly, the recombinant transfer vector plasmid pVL1392-dORF2 was produced and then transfected with linearized BestBac™ 2.0 (Av-cath/chiA) parental baculovirus DNA (Expression Systems LLC, Davis, CA) to generate recombinant baculovirus AcBestBac-dORF2. Both AcBactoBac-dORF2 and AcBestBac-dORF2 were routinely amplified on Sf-9 cells grown in ESF921 serum-free medium (Expression Systems LLC, Davis, CA) at 27° C. Virus titers were determined using antibodies against gp64.

The general parameters used for inoculation (by AcBactoBac-dORF2 or AcBestBac-dORF2) and harvest of the cell culture supernatant. During the optimization phase, twelve parameters were tested and performed as follows. 1^{st} day: Seed four 500 mL cultures of High Five cells in ESF921 at 1×10⁶ cells/mL. 2^{nd} day: Infect one culture at a multiplicity of infection (MOI)=0.1 and one culture at an MOI=3.0 with HEV-573C (BestBac). Infect one culture at an MOI=0.1 and one at an MOI=3.0 with HEV-573C (Bacmid). Incubate cultures shaking for two hours. For each bottle, transfer 100 mL culture to each of the four 250 mL flasks. Determine viable cell count and percent viability daily. Stain cells for gp64 expression on 1 and 2 days post-infection (dpi). At 5, 6, and 7 dpi, pellet cells from 100 mL of each culture condition and sterile transfer supernatant to two 50 mL tubes for each culture. Store the pellet at −80° C. Store supernatant samples at 4° C.

Subsequently, the High Five cells at 2 million cells per mL were inoculated at a multiplicity of infection of 3 viruses per cell (MOI=3). At 6 dpi, the supernatant of the cells was collected for purification. Ultracentrifugation purification of QINs expressed from variants of ORF2 constructs was conducted. The QINs were collected and purified through multiple centrifugation steps, including CsCl equilibrium density gradient ultracentrifugation. The purified QINs were resuspended in 10 mM potassium-MES buffer, pH 6.2, and stored at 4° C. The protein concentration and purity of fractions showing a positive signal of QIN in the ELISA test were determined via UV spectrometry (A280) using a NanoDrop™ spectrophotometer.

### Building Construct of Chimeric Nanoparticles QIN-GMI:

The GMI epitope, MSDTALIFTLAWNVK (SEQ ID NO: 3), was inserted into the position 498-506 aa of QIN, dORF2 (i.e., capsid subunit of the QIN herein) (obtained from e.g., but not limited to, crystal structure analysis of the capsid protein [Hepatitis E virus (strain Myanmar)] Sequence ID: 2ZZQ_A (SEQ ID NO: 1); other known sequences of ORF2 also can be used herein, such as those described in the U.S. patent No. US20190031720 A1) to obtain QIN-GMI represented by SEQ ID NO: 4, and synthesized by Integrated DNA Technologies, Inc. (IDT) after sequence optimization for the best expression in insect cells. The following procedures are similar to those described above for the building construct of QIN. In short, the synthesized DNA fragment was cloned into the BestBac Bacmid vector (Expression Systems, LLC, Davis, CA, USA). The mutated ORF2 proteins were expressed in High Five cells at MOI-3 and collected at 6 dpi followed by multiple steps of density gradient ultracentrifugation (referring to Li et al., 2005, J Virol 79 (20): 12999-13006) [2]. The purified chimeric nanoparticles QIN-GMI were resuspended in 10 mM potassium-MES buffer, pH 6.2, and stored at 4° C.

### Transmission Electron Microscopy:

The purified QIN and QIN-GMI were loaded onto a glow-discharged, carbon-coated EM grid and stained with 2% uranyl acetate and examined under a JOEL JEM-1230 transmission electron microscope at the magnifications of 30,000. The images were recorded on a CCD camera (TVIPS Gauting, Germany).

TABLE 1
Amino acid sequence list

| Name | SEQ ID NO: | Amino Acid Sequence |
|---|---|---|
| PORF2- | 1 | 1 MFIQPTPSPP MSPLRPGLDL VFANPPDHSA PLGVTRPSAP |
| QIN | | 41 PLPHVVDLPQ LGPRRAVAPA HDTPPVPDVD SRGAILRRQY |
| (2ZZQ_A) | | 81 NLSTSPLTSS VATGTNLVLY AAPLSPLLPL QDGTNTHIMA |
| | | 121 TEASNYAQYR VARATIRYRP LVPNAVGGYA ISISFWPQTT |
| | | 161 TTPTSVDMNS ITSTDVRILV QPGIASELVI PSERLHYRNQ |
| | | 201 GWRSVETSGV AEEEATSGLV MLCIHGSPVN SYTNTPYTGA |
| | | 211 LGLLDFALEL EFRNLTPGNT NTRVSRYSST ARHRLRRGAD |
| | | 281 GTAELTTTAA TRFMKDLYFT STNGVGEIGR GIALTLFNLA |
| | | 321 DTLLGGLPTE LISSAGGQLF YSRPVVSANG EPTVKLYTSV |
| | | 361 ENAQQDKGIA IPHDIDLGES RVVIQDYDNQ HEQDRPTPSP |
| | | 401 APSRPFSVLR ANDVLWLSLT AAEYDQSTYG SSTGPVYVSD |
| | | 441 SVTLVNVATG AQAVARSLDW TKVTLDGRPL STIQQYSKTF |
| | | 481 FVLPLRGKLS FWEAGTTKAG YPYNYNTTAS DQLLVENAAG |
| | | 521 HRVAISTYTT SLGAGPVSIS AVAVLAPHSA LA |
| modified | 2 | 1 MAVAPAHDTP PVPDVDSRGA ILRRQYNLST SPLTSSVATG |
| PORF2- | | 41 TNLVLYAAPL SPLLPLQDGT NTHIMATEAS NYAQYRVARA |
| QIN | | 81 TIRYRPLVPN AVGGYAISIS FWPQTTTTPT SVDMNSITST |
| | | 121 DVRILVQPGI ASELVIPSER LHYRNQGWRS VETSGVAEEE |
| | | 161 ATSGLVMLCI HGSPVNSYTN TPYTGALGLL DFALELEFRN |
| | | 201 LTPGNTNTRV SRYSSTARHR LRRGADGTAE LTTTAATRFM |
| | | 241 KDLYFTSTNS VGEIGRGIAL TLFNLADTLL GGLPTELISS |
| | | 281 AGGQLFYSRP VVSANGEPTV KLYTSVENAQ QDKGIAIPHD |
| | | 321 IDLGESRVVI QDYDNQHEQD RPTPSPAPSR PFSVLRANDV |
| | | 361 LWLSLTAAEY DQSTYGSSTG PVYVSDSVTL VNVATGAQAV |
| | | 401 ARSLDWTKVT LDGRPLSTIQ QYSKTFFVLP LRGKLSFWEA |
| | | 441 GTTKAGYPYN YNTTASDQLL VECAAGHRVA ISTYTTSLGA |
| | | 481 GPVSISAVAV LAPHSALA |
| GMI | 3 | MSDTALIFTL AWNVK |
| epitope | | |
| PORF2- | 4 | 1 MFIQPTPSPP MSPLRPGLDL VFANPPDHSA PLGVTRPSAP |
| QIN-GMI | | 41 PLPHVVDLPQ LGPRRAVAPA HDTPPVPDVD SRGAILRRQY |
| | | 81 NLSTSPLTSS VATGTNLVLY AAPLSPLLPL QDGTNTHIMA |
| | | 121 TEASNYAQYR VARATIRYRP LVPNAVGGYA ISISFWPQTT |
| | | 161 TTPTSVDMNS ITSTDVRILV QPGIASELVI PSERLHYRNQ |
| | | 201 GWRSVETSGV AEEEATSGLV MLCIHGSPVN SYTNTPYTGA |
| | | 241 LGLLDFALEL EFRNLTPGNT NTRVSRYSST ARHRLRRGAD |
| | | 281 GTAELTTTAA TRFMKDLYFT STNGVGEIGR GIALTLFNLA |
| | | 321 DTLLGGLPTE LISSAGGQLF YSRPVVSANG EPTVKLYTSV |
| | | 361 ENAQQDKGIA IPHDIDLGES RVVIQDYDNQ HEQDRPTPSP |
| | | 401 APSRPFSVLR ANDVLWLSLT AAEYDQSTYG SSTGPVYVSD |
| | | 441 SVTLVNVATG AQAVARSLDW TKVTLDGRPL STIQQYSKTF |
| | | 481 FVLPLRGKLS FWEAGTTMSD TALIFTLAWN VKTTASDQLL |
| | | 521 VECAAGHRVA ISTYTTSLGA GPVSISAVAV LAPHSALA |

Animal test (referring to Do et al., 2021, Can J Vet Res 85(2):93-100)^{[2]};

Protocol for the animal trial carried out in this study was reviewed and approved by the Institutional Animal Care and Use Committee (IACUC) at Reber Genetics, Co. Ltd. and the study was carried out at the animal facility of GMP plant in Reber Genetics, Co., Ltd. 21-day-old (+/−1 day) healthy pigs that tested negative for PCV2, porcine reproductive and respiratory syndrome virus (PRRSV), and classical swine fever virus (CSFV) was selected for this study.

This study has three trial groups: Group A, Group B, and Group C. In Group A: 4 piglets were used as placebo group; Group B: 5 piglets were vaccinated with CIRCOQ and orally fed QIN daily at the amount of 20 μg per kg from 3 to 6 weeks old; and Group C: 5 piglets were vaccinated with CIRCOQ only. All piglets were then challenged four weeks later (i.e. 10 weeks old) with PCV2 virus. The immune response and weight gain were analyzed as the factors of the immune modulator. The measurement of weight gain, IgG and IgA levels, and the statistical analysis were conducted.

All experimental pigs had an ear tagged, and numbers were clicked to be tracked individually. Afterward, pigs in each group were kept in the same house in different pens and were cared for in the same manner. Clinical signs, including respiratory failure, dermatitis, stunted growth, and death, were monitored daily. The productivity indicators, including average weight gain (AWG) and average daily weight gain (ADWG), were calculated based on the time the experiment began at 21 d old and ended at 182 d old.

Thirty serum samples were randomly collected from each group at 0, 14, 28, 56, 84, 140, and 161 d after vaccination and were tested with a commercial PCV2 enzyme-linked immunosorbent assay (ELISA) Kit (SK105; BioChek, Reeuwijk, Netherlands) to determine antibody response and levels of PCV2 viremia. Serum samples were considered positive for PCV2 antibody if the sample-to-positive (S/P) ratio was higher than 0.4, in accordance with the manufacturer's instructions.

### Statistical Analysis

Continuous data (Ct value of PCV2 qRT-PCR, serology IgG, and IgA) were analyzed using 1-way analysis of variance (ANOVA), followed by Tukey's multiple comparison tests at each time point. Fisher's exact and Chi-square tests were used for discrete data (clinical signs such as respiratory failure, stunted growth, dermatitis, and death). A value of P<0.05 was considered significant.

All the above-cited patents and literature are expressly and entirely incorporated herein by reference.

### Selected Genes in the Present Examples:

### Innate Immunity: TLR8/TLR7, LY96, TLR4, CD14

Toll-like receptors (TLRs) acting as important pathogen recognition receptors (PRRs) in host are key molecules for innate immune system, inflammatory response and adaptive immunity. Among them, TLR8 and TLR7, which share a high degree of structural similarity, recognize viral single-stranded RNA (ssRNA). Another toll-like receptor, TLR5, regarded as therapeutic potential through its innate immunity to treat Alzheimer's disease (AD).

Lymphocyte antigen 96 (LY96), also known as “Myeloid Differentiation factor 2 (MD-2)”, and TLR4 are related to innate immunity's LPS-LY96 (MD2)-TLR4 pathway. LY96 together with Toll-like receptor 4 (TLR4), binds lipopolysaccharide (LPS) with high affinity, inducing the formation of the activated homodimer LPS-LY96 (MD2)-TLR4, leading to the activation of downstream signaling of cytokine and chemokine production, and initiation of inflammatory and immune responses.

CD14, is a glycosylphosphatidylinositol (GPI)-anchored receptor known to serve as a co-receptor working with TLR4 and facilitating cellular responses to low doses of lipopolysaccharide (LPS).

The development of chronic infections and cancer is facilitated by a variety of immune subversion mechanisms, such as the production of anti-inflammatory cytokines, induction of regulatory T (Treg) cells, and expression of immune checkpoint molecules, including CTLA-4 and PD-1. CTLA-4, expressed on T cells, interacts with CD80/CD86, thereby limiting T-cell activation.

### Tumor Immunity: ICAM1, ITGAM, NFKBIA

Intracellular adhesion molecule 1 (ICAM1) has been implicated in a variety of immune and inflammatory responses and the formation of tertiary lymphoid structure (TLS) within the tumor microenvironment. The variations of ITGAM gene, which encodes for the CD11b chain of the αMB2 (Mac-1; CD11b/CD18) integrin, is one of the strongest genetic risk factors for autoimmune diseases such as systemic lupus erythematosus (SLE). NFKBIA, encoding nuclear factor of k-light polypeptide gene enhancer in B-cells inhibitor-α, was proposed as an inhibitor of the EGFR-signaling pathway which is related to tumorigenesis.

### Cytokine/Humoral Immunity/Inflammatory Response: C3, MX1, CCL2, CXCL10, CXCL8, CCL5, CD40, CD4

Complement protein C3 is required for the effective induction of humoral and cellular adaptive immune responses to vaccination with M2e-based or HA-based vaccines. MX1 is a GTPase, in part, the infected host's antiviral response induced by interferons (type I and III). Viral proteins, like polymerase basic protein 2 (PB2) and nucleoprotein (NP), were suggested to be the direct targets of MX1. Inhibition of the PB2-NP interaction is an active process requiring enzymatically active MX1. The chemokine CCL2, also called “Monocyte chemoattractant protein-1” (MCP-1), and its main chemokine receptor CCR2 have been implicated in the pathogenesis of several different disease processes, including vascular permeability and attraction of immune cells during metastasis, several different neurological disorders, autoimmune disease, obesity, and atherosclerosis. The involvement of CCL2 signaling in multiple diseases renders it an attractive therapeutic target. The antimicrobial gene, CXCL-10, encodes a chemokine of the CXC subfamily and ligand for the receptor CXCR3. Binding of CXCL-10 to CXCR3 results in pleiotropic effects, including stimulation of monocytes, natural killer and T-cell migration, and modulation of adhesion molecule expression. The ligand CXCL8 and its chemokine receptors CXCR1/2 are essential for the activation and trafficking of inflammatory mediators and tumor progression and metastasis. The CXCL8-CXCR1/2 signaling axis is involved in the pathogenesis of several diseases, including chronic obstructive pulmonary diseases (COPD), asthma, cystic fibrosis, and cancer.

CCL5 is a Cytotoxic CD8⁺ T cell that has been considered one of the main populations of effector immune cells in antitumor immunity; however, the absence of CD8⁺ T cells in the central tumor area has become a significant obstacle for solid tumor immunotherapy, particularly for colorectal cancer (CRC). Prior research demonstrated that CCL5-deficiency delayed tumor growth and metastasis by facilitating CD8⁺ T cells to accumulate into tumor sites in CRC mouse models.

CD40 is a promising therapeutic target for cancer immunotherapy. CD40 agonists have demonstrated some antitumor activity and a manageable toxicity profile in patients with advanced solid malignancies.

CD4⁺ T cells are recognized mainly as the orchestrators of immune responses, being able to differentiate into distinct T helper cell populations based on differentiation signals, transcription factor expression, cytokine secretion, and specific functions.

Interleukin 1β (IL1B) is a pro-inflammatory cytokine important for both normal immune responses and chronic inflammatory diseases.

The cytotoxic T cell (CTL) response is determined by the peptide repertoire presented by the HLA class I molecules of an individual. Senescent cells accumulate in human tissues during ageing and contribute to age-related pathologies. It was proposed that HLA-E expression is induced by senescence-associated secretary phenotype-related pro-inflammatory cytokines, and is regulated by p38 MAP kinase signaling in vitro.

STAT1 along with STAT2 and IRF9 are the interferon-stimulated genes responsible for signal transduction downstream both type I and III IFN receptors. The transcriptional regulation of their expression represents an important feedback loop regulating IFN signaling.

### Heme Catabolism: HMOX1

Heme oxygenase, an essential enzyme in heme catabolism, cleaves heme to form biliverdin, which is subsequently converted to bilirubin by biliverdin reductase, and carbon monoxide, a putative neurotransmitter. Heme oxygenase occurs as 2 isozymes, an inducible heme oxygenase-1 (HMOX1) and a constitutive heme oxygenase-2 (HMOX2).

### Intestine Immunity: CCR6 is Suppressed by QIN

The intestinal lamina propria (LP) contains antigen-presenting cells with features of dendritic cells and macrophages, collectively called mononuclear phagocytes (MNPs). Association of MNPs with the epithelium is thought to play an essential role in multiple facets of intestinal immunity, including imprinting MNPs with the ability to induce IgA production, inducing the expression of gut-homing molecules on T cells, facilitating the capture of luminal antigens and microbes, and subsequent immune responses in the mesenteric lymph node (MLN). CCR6, the only known receptor for CCL20, was required for MNPs to associate with the epithelium. These findings identify a crucial role for CCR6 in promoting LP-MNPs to associate with the intestinal epithelium in the steady state to perform multiple functions promoting gut immune homeostasis.

AQP1: This gene encodes a small integral membrane protein with six bilayer spanning domains that functions as a water channel protein. This protein permits passive transport of water along an osmotic gradient. This gene is a possible candidate for disorders involving imbalance in ocular fluid movement.

IL6: Interleukin 6 (IL-6) has a broad effect on cells of the innate and adaptive immune system and those not of the immune system and often displays hormone-like characteristics that affect homeostatic processes. IL-6 has context-dependent pro- and anti-inflammatory properties and is now regarded as a prominent target for clinical intervention.

IL23A: Risankizumab is a fully human monoclonal antibody that selectively targets interleukin (IL)-23A, interfering with the IL-23/17 axis that plays a crucial role in keratinocyte proliferation. In 2019, risankizumab was approved globally for the treatment of moderate-to-severe psoriasis. Drugs blocking the IL-23 pathway are the most recently approved treatment for psoriasis, and risankizumab seems to be the most effective one among the three IL-23 blockers approved.

DDX58: Interestingly, some of the more recent innate immune gene associations with psoriasis have been found in interferon and antiviral response genes. These genes are DDX58 (DEAD (Asp-Glu-Ala-Asp) box polypeptide 58), which encodes the protein RIG-I, and IFIH1, which encodes the protein MDA5. Both RIG-I and MDA5 are intracellular, innate immune sensors, critical for detection of double stranded RNAs. RIG-I specifically recognizes shorter RNAs containing 5′ triphosphate moieties and blunt-end base pairing at the 5′ end, whereas MDA5 is thought to recognize longer RNA structures. Both are important for recognition of dsRNA viruses or viruses that utilize dsRNA intermediates of replication, and the subsequent elicitation of a type-I interferon response.

### Experimental Results

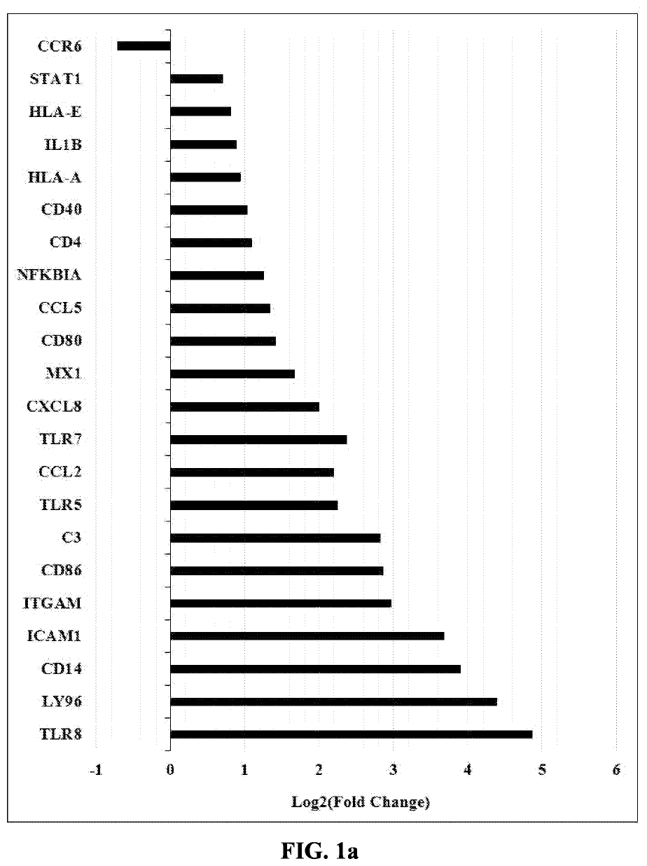

Please refer to the FIGS. 1a and 1b, wherein FIG. 1a illustrates the expression of 22 selected genes affected by QINs in qPCR analysis with QIAGEN plates, and FIG. 1b illustrates the expression of 4 selected genes affected by QINs in individual qPCR analysis. It can be found that most of the total 26 genes displayed a somewhat consistent pattern, wherein except CCR6 and AQP1 was a little bit suppressed in THP-1 monocytes, the remaining genes of STAT1, HLA-E, UL1B, HLA-A, CD40, CD4, NFKBIA, CCL5, CD80, MX1, CXCL8, TLR7, CCL2, TLR5, C3, CD86, ITGAM, ICAM1, CD14, LY96, TLR8, TLR4, HMOX1 and CXCL10 were stimulated/activated by QINs.

### Protein-Protein Interaction Analysis

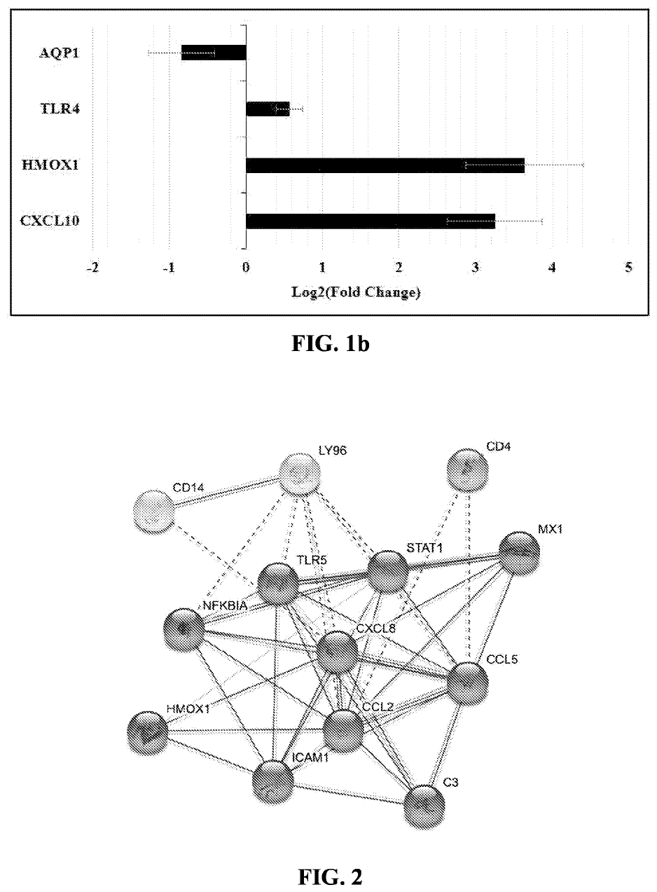

Please refer to FIG. 2, which illustrates among all selected genes expression affected by QINs, the relationship of 13 selected genes interacted with each other shown in the generated Protein-Protein Interaction Network. The protein-protein interaction (PPI) map of the 13 highly expressed genes shown in FIGS. 1a and 1b was generated with STRING toolkit (https://version11.string-db.org/cgi/input.pl?sessionId=uoELhwcyqUyX&input_page_show_search=on) to analyze the genes stimulated by QINs. The different colors were indicative of clustering (grouping) for similarity in interaction. According to the protein-protein interaction map, 10 genes, including HMOX1 are closely related to each other.

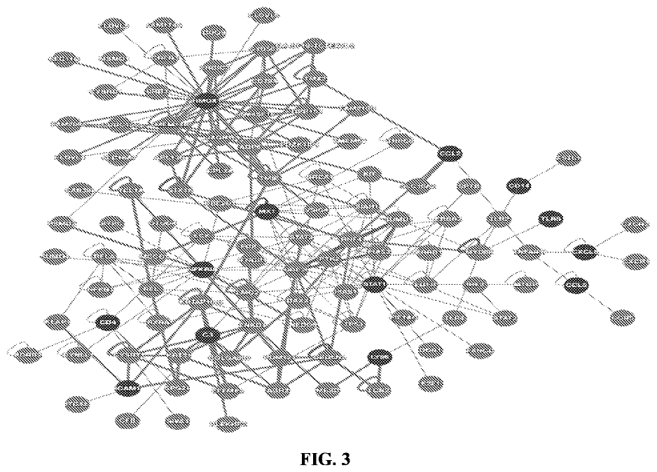

Please further refer to FIG. 3, which illustrates Protein-Protein Interaction Network expanded from the 13 genes stimulated by QINs in FIG. 2. The 13 genes stimulated by QINs shown in FIG. 2 were further expanded to other related genes using the current knowledge of Protein-Protein Interaction and generated another map with 113 interactors in total. Among them, 19 genes are related to positive regulation of transcription from RNA polymerase II promoter; 16 genes to Signal transduction; 14 genes to Apoptotic process; 14 genes to transcription, DNA template; 12 to Innate immune response. Please see Table 2 below for details.

TABLE 2
Gene ontology (GO) annotation of the QIN-GMI
network for the 13 highly expressed genes

| Biological Process | Count | % | P Value |
|---|---|---|---|
| Positive regulation of transcription from | 19 | 23.5 | 5.44E−08 |
| RNA polymerase II promoter | | | |
| Signal transduction | 16 | 19.8 | 5.91E−05 |
| Inflammatory response | 15 | 18.5 | 3.52E−10 |
| Apoptotic process | 14 | 17.3 | 4.41E−07 |
| Transcription, DNA-templated | 14 | 17.3 | 5.33E−02 |
| Innate immune response | 12 | 14.8 | 1.31E−06 |
| Positive regulation of transcription, DNA- | 11 | 13.6 | 4.52E−05 |
| templated | | | |
| Positive regulation of gene expression | 10 | 12.4 | 1.20E−06 |
| Negative regulation of transcription from | 10 | 12.4 | 2.64E−03 |
| RNA polymerase II promoter | | | |
| Cellular response to lipopolysaccharide | 9 | 11.1 | 1.95E−08 |
| Viral process | 9 | 11.1 | 3.00E−05 |
| Negative regulation of apoptotic process | 9 | 11.1 | 5.37E−04 |
| Lipopolysaccharide-mediated signaling | 8 | 9.9 | 4.49E−11 |
| pathway | | | |
| Cell surface receptor signaling pathway | 8 | 9.9 | 1.30E−04 |
| Positive regulation of GTPase activity | 8 | 9.9 | 8.37E−03 |

### QIN vs QIN-GMI

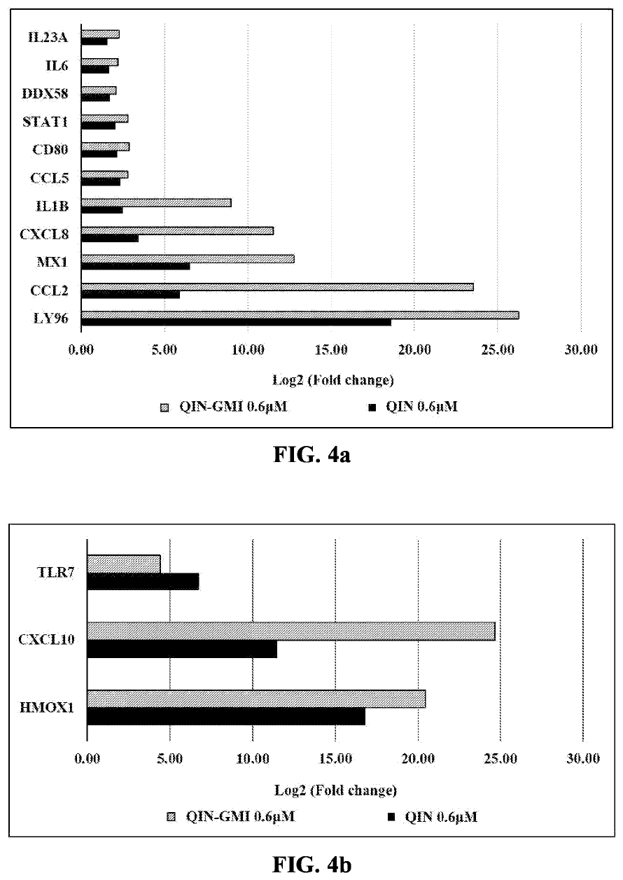

Please refer to the FIGS. 4a and 4b, wherein FIG. 4a illustrates the expression of 11 selected genes affected by QIN and QIN-GMI in qPCR analysis with QIAGEN plates and FIG. 4b illustrates the expression of 3 selected genes affected by QIN and QIN-GMI in individual qPCR analysis. It can be found that, in general, the genes stimulated by QIN-GMI are similar to QIN alone, and all the 14 selected genes matched this criterion. Among them, 13 genes (50% of the 26 genes in the QIN list) of the QIN-GMI-treated condition were in the same direction as the QIN-treated condition. One gene, TLR7, was slightly in the opposite direction.

### GMI vs QIN vs QIN-GMI

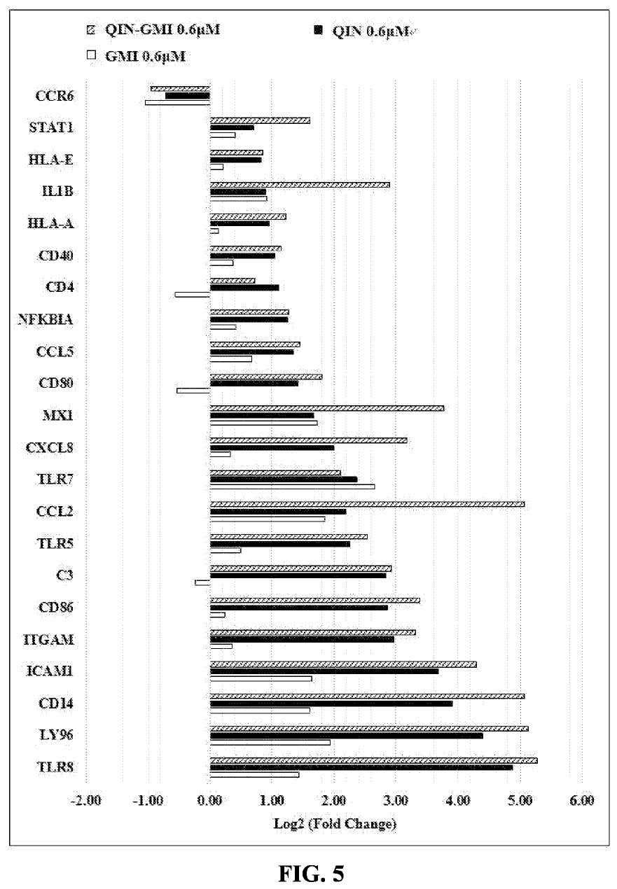

Further refer to the FIG. 5, which illustrates the expression of 22 selected genes affected by GMI, QIN, and QIN-GMI in qPCR gene analysis with QIAGEN plates. It can be found that, in general, the genes stimulated by GMI, QIN, and QIN-GMI in a similar trend, and all the 22 selected genes matched this criterion. Among them, 19 genes (80% of the genes) of the GMI-treated condition were in the same trend as the QIN- and QIN-GMI-treated condition. Three genes, including CD4, CD80, and CD3, were affected in the opposite direction. Surprisingly, the QIN alone showed similar gene profiles with GMI and QIN-GMI, which suggests its potential role as an immune modulator.

### Animal Test Results:

Based on the experimental results, QIN was further used as an oral immune modulator in the PCV2 subunit vaccinated piglets with improved immune response and weight gain.

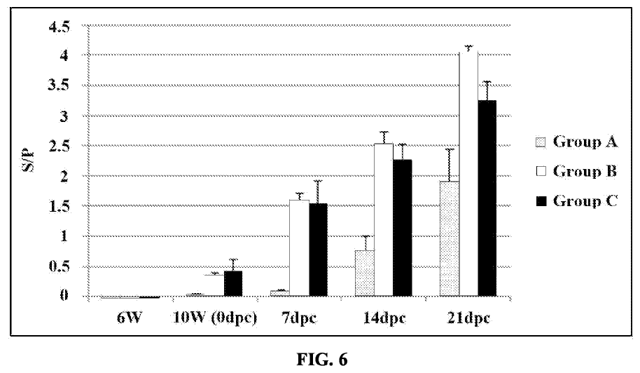

Please refer to Table 3 and FIG. 6. It can be found that the average weight gain (AWG) or average daily weight gain (ADWG) of pigs in group B vaccinated with PCV2 subunit vaccine, CIRCOQ, and orally fed QIN daily was the highest, followed by pigs in Group C vaccinated with CIRCOQ only, and finally pigs in group A with placebo is the lowest (Table 3). The same trend was found in serum PCV2-specific IgG response (FIG. 6). In comparison, there was no difference in the IgA measurement (data not shown).

TABLE 3
Growth and body weight of pigs treated with placebo,
CIRCOQ plus oral QIN and CIRCOQ only, respectively

| | 6 weeks | 10 weeks | 13 weeks | ADWG |
|---|---|---|---|---|
| | (kg) | (kg) | (kg) | (g) |
| Group A: | 8.73 | 17.88 | 21.95 | 473.0952 |
| placebo | | | | |
| Group B: | 9.20 | 19.40 | 25.62 | 586.4286 |
| CIRCOQ + | | | | |
| oral QIN | | | | |
| Group C: | 8.57 | 18.16 | 24.08 | 553.8571 |
| CIRCOQ | | | | |

### Discussion

The original rationale of the research was to use QIN as an oral delivery carrier for an immune regulator, GMI. Besides QIN-GMI, QIN alone can stimulate similar gene profiles to GMI in HTP-1 cells. From the protein-protein interaction network generated from the 13 highly expressed genes stimulated by QIN, only 9 out of 113 affected genes related to the viral process (Table 2). The finding could indicate QIN alone could be a potential immune modulator without triggering a solid viral immune response. However, a similar gene profile with more vital stimulation by GMI peptide inserted QIN (i.e., QIN-GMI) could be explained by the multivalent QIN, which contains 60 copies of GMI peptide on its surface. The stronger stimulation of gene profiles related to innate/adaptive immune response could indicate the dose of the QIN only. QIN-GMI lower than GMI alone is required to function as an immune modulator. The immune-modulator effect of oral-fed QIN was further verified by the animal test on piglets vaccinated with the PCV2 subunit vaccine, CIRCOQ, by showing better average weight gain and higher IgG titer.

### CONCLUSION

As described above, a chimeric QIN-GMI can stimulate a higher gene expression profile than QIN only and GMI only without stimulating strong viral immune response in THP-1 monocytes. Surprisingly, the QIN alone also showed less than but a similar trend as QIN-GMI in gene analysis. The results show that QIN alone was used as an oral immune modulator in the animal test on piglets vaccinated with PCV2 subunit vaccine, CIRCOQ, showing better average weight gain and higher IgG titer. Thus, it can prove that QIN as an immune modulator (QIN-IM) and even QIN-GMI positively affect the PCV2 subunit vaccine.

Without further elaboration, it is believed that one skilled in the art can, based on the above description, utilize the present invention to its fullest extent. Therefore, the above specific examples are to be construed as merely illustrative and not limitative of the remainder of the disclosure in any way whatsoever.

### REFERENCES

1. Li T-C, Takeda N, Miyamura T, Matsuura Y, Wang J C Y, Engvall H, Hammar L, Xing L, Cheng RH: Essential elements of the capsid protein for self-assembly into empty virus-like particles of hepatitis E virus. J Virol 2005, 79 (20): 12999-13006.
2. Do D T, Tran K D V, Quach A T, Lee D, Chang F C, Wu C P, Tat T N, Chae C: A comparative efficacy test of 1 versus 2 doses of CIRCOQ PCV2 subunit vaccine against naturally occurring PCV2-type d in piglets with high maternally derived antibodies (MDAs) on a Vietnamese swine farm. Can J Vet Res 2021, 85 (2): 93-100.

What is claimed is:

1. A method of enhancing an immune response in a subject in need thereof, the method comprising administering an effective amount of an antigen with an immune modulator to the subject, wherein the immune modulator is a quaternary immunomodulatory nanoparticle (QIN).

2. The method of claim 1, wherein the QIN is further coupled with an epitope of a Ganoderma microsporum immunomodulatory (GMI) protein.

3. The method of claim 2, wherein the epitope of the GMI protein is coupled with QIN by inserting into a capsid subunit of the QIN.

4. The method of claim 2, wherein the epitope of the GMI protein has a sequence represented by SEQ ID NO: 1.

5. The method of claim 1, wherein the QIN is orally administered to the subject.

6. The method of claim 1, wherein the subject comprises an animal species selected from the group consisting of mouse, rat, rabbit, dog, pig, sheep, cow, and primate.

7. The method of claim 1, wherein the antigen is selected from a group consisting of a tumor antigen, a virus antigen, a bacterial antigen, an autoantigen, and a fungal antigen.

8. The method of claim 7, wherein the antigen is a viral antigen.

9. The method of claim 8, wherein the virus antigen is a porcine circovirus type 2 (PCV2) antigen.

10. The method of claim 9, wherein the PCV2 antigen is administered to the subject via a PCV2 vaccine.

11. A chimeric nanoparticle comprising a QIN coupled and an epitope of a GMI protein.

12. The chimeric nanoparticle of claim 11, wherein the epitope of the GMI protein is coupled with the QIN by inserting into a capsid subunit of the QIN.

13. The chimeric nanoparticle of claim 11, wherein the epitope of the GMI protein has a sequence represented by SEQ ID NO: 1.

14. A pharmaceutical composition comprising a QIN and a pharmaceutically acceptable carrier, vehicle, or diluent.

15. The pharmaceutical composition of claim 14, wherein the QIN is further coupled with an epitope of a GMI protein.

16. The pharmaceutical composition of claim 15, wherein the epitope of the GMI protein is coupled with the QIN by inserting into a capsid subunit of the QIN.

17. The pharmaceutical composition of claim 15, wherein the epitope of the GMI protein has a sequence represented by SEQ ID NO: 1.

18. A use of the pharmaceutical composition of claim 15 in the manufacture of a medicament for treating a subject suffering from cancer or virus infection.


SEQUENCE LISTING (4 sequences)

SEQ ID NO: 1 - protein, 552 aa, synthetic construct
MFIQPTPSPPMSPLRPGLDLVFANPPDHSAPLGVTRPSAPPLPHVVDLPQLGPRRAVAPAHDTPPVPDVDSRGAILRRQYNLSTSPLTSSVATGTNLVLYAAPLSPLLPLQDGTNTHIMATEASNYAQYRVARATIRYRPLVPNAVGGYAISISFWPQTTTTPTSVDMNSITSTDVRILVQPGIASELVIPSERLHYRNQGWRSVETSGVAEEEATSGLVMLCIHGSPVNSYTNTPYTGALGLLDFALELEFRNLTPGNTNTRVSRYSSTARHRLRRGADGTAELTTTAATRFMKDLYFTSTNGVGEIGRGIALTLFNLADTLLGGLPTELISSAGGQLFYSRPVVSANGEPTVKLYTSVENAQQDKGIAIPHDIDLGESRVVIQDYDNQHEQDRPTPSPAPSRPFSVLRANDVLWLSLTAAEYDQSTYGSSTGPVYVSDSVTLVNVATGAQAVARSLDWTKVTLDGRPLSTIQQYSKTFFVLPLRGKLSFWEAGTTKAGYPYNYNTTASDQLLVENAAGHRVAISTYTTSLGAGPVSISAVAVLAPHSALA

SEQ ID NO: 2 - protein, 498 aa, synthetic construct
MAVAPAHDTPPVPDVDSRGAILRRQYNLSTSPLTSSVATGTNLVLYAAPLSPLLPLQDGTNTHIMATEASNYAQYRVARATIRYRPLVPNAVGGYAISISFWPQTTTTPTSVDMNSITSTDVRILVQPGIASELVIPSERLHYRNQGWRSVETSGVAEEEATSGLVMLCIHGSPVNSYTNTPYTGALGLLDFALELEFRNLTPGNTNTRVSRYSSTARHRLRRGADGTAELTTTAATRFMKDLYFTSTNSVGEIGRGIALTLFNLADTLLGGLPTELISSAGGQLFYSRPVVSANGEPTVKLYTSVENAQQDKGIAIPHDIDLGESRVVIQDYDNQHEQDRPTPSPAPSRPFSVLRANDVLWLSLTAAEYDQSTYGSSTGPVYVSDSVTLVNVATGAQAVARSLDWTKVTLDGRPLSTIQQYSKTFFVLPLRGKLSFWEAGTTKAGYPYNYNTTASDQLLVECAAGHRVAISTYTTSLGAGPVSISAVAVLAPHSALA

SEQ ID NO: 3 - protein, 15 aa, synthetic construct
MSDTALIFTLAWNVK

SEQ ID NO: 4 - protein, 558 aa, synthetic construct
MFIQPTPSPPMSPLRPGLDLVFANPPDHSAPLGVTRPSAPPLPHVVDLPQLGPRRAVAPAHDTPPVPDVDSRGAILRRQYNLSTSPLTSSVATGTNLVLYAAPLSPLLPLQDGTNTHIMATEASNYAQYRVARATIRYRPLVPNAVGGYAISISFWPQTTTTPTSVDMNSITSTDVRILVQPGIASELVIPSERLHYRNQGWRSVETSGVAEEEATSGLVMLCIHGSPVNSYTNTPYTGALGLLDFALELEFRNLTPGNTNTRVSRYSSTARHRLRRGADGTAELTTTAATRFMKDLYFTSTNGVGEIGRGIALTLFNLADTLLGGLPTELISSAGGQLFYSRPVVSANGEPTVKLYTSVENAQQDKGIAIPHDIDLGESRVVIQDYDNQHEQDRPTPSPAPSRPFSVLRANDVLWLSLTAAEYDQSTYGSSTGPVYVSDSVTLVNVATGAQAVARSLDWTKVTLDGRPLSTIQQYSKTFFVLPLRGKLSFWEAGTTMSDTALIFTLAWNVKTTASDQLLVECAAGHRVAISTYTTSLGAGPVSISAVAVLAPHSALA